(19) United States
(12) Patent Application Publication
(10) Pub. No.: US 2026/0284304 A1
(43) Pub. Date: Sep. 24, 2026

(54) INVERTED CAM-OPERATED FORCE-DRIVEN INFUSION PUMPING MECHANISM

(71) Applicant: CareFusion 303, Inc., San Diego, CA (US)

(72) Inventors: Richard O’CONNOR, Limerick (IE); Maurya Sundar VIJAYA KUMAR, Limerick (IE)

(21) Appl. No.: 19/084,660

(22) Filed: Mar. 19, 2025

(51) Int. Cl. A61M 5/145; A61M 5/142

(52) U.S. Cl. CPC A61M 5/1452; A61M 5/14228; A61M 2005/14533

(57) ABSTRACT

Disclosed is an infusion pump that utilizes force-driven infusion compression elements that are operated by an inverted cam profile. The compression elements, or pump fingers, are driven by force generating components, each exerting a compression force on a respective compression element to cause the compression element to compress the fluid tubing against a support surface. Each cam of the pump is configured to, when rotated, periodically apply a counterforce to a respective compression element to counteract the compression force being applied to the compression element by the force generating component and to lift the respective compression element away from compressing the fluid tubing. As the cams are collectively rotated, the compression forces exerted on the compression elements, counteracted by the periodic lifting of the respective compression elements, create a sinusoidal compression force against the fluid tubing to displace the fluid within the fluid tubing in a downstream direction of flow.

### TECHNICAL FIELD

The present disclosure relates generally to intravenous infusion pump mechanisms. In particular, the present disclosure relates to operation of an infusion pump on an infusion set.

### BACKGROUND

In the medical field, infusion pumps are used often to administer accurate amounts of infusates from an external bag to a patient via an intravenous (IV) administration set. The IV administration set is used to administer fluids, medications, or blood products directly into the patient's bloodstream. It typically consists of a plastic or flexible tubing with connectors on either end—one end attaches to a fluid source such as a saline bag or medication vial, while the other end connects to a needle or catheter inserted into a vein. The IV administration set includes a pumping segment—a section of tubing in the IV administration set—that loads into the infusion pump and passes through the pump's rollers or squeeze mechanism. As the pump compresses and releases the tubing of the pumping segment in a rhythmic motion, it generates a pulsatile flow, effectively propelling fluid through the tubing of the IV administration set and into the patient's bloodstream.

### SUMMARY

Existing volumetric infusion pumps operate by compressing an elastic segment of the IV administration set against a platen to deliver fluid. The mechanics of the pumping mechanism rely on displacement of numerous pump fingers to displace the fluid within the set according to a predetermined tightly synchronized pumping cycle. As the position of the fingers progress through the cycle there is a transition zone where neither finger is occluding the set. For example, a first finger may start to move up while a second finger is moving downward. This makes the rate accuracy very sensitive at low speed, as well as increasing sensitivity to back pressure.

The present disclosure addresses the aforenoted problems by providing an infusion pump pumping mechanism that utilizes force-driven infusion pumping elements/fingers that are operated by an inverted cam profile. In this regard, each pumping finger is preloaded with a spring element to apply a default compression force to the IV administration set loaded within the pump. The cam operates to lift each finger from the set instead of conventionally pushing the fingers into the set. In this regard, the compression of the IV infusion set being less sensitive to variation in the set geometry and mechanism tolerances, such that there is margin in the spring force utilized to occlude the set. Further benefits include more consistent flow rates being provided across all rotational speeds, potentially removing the need to complex linearizing algorithms to compensate at low flow rates. Sensitivity to back pressure, in particular at low flow rates, may also be reduced.

According to various implementations, an infusion pump comprises: a plurality of compression elements configured to compress a fluid tubing filled with a fluid against a rigid tubing support surface when the fluid tubing is received by the infusion pump; a plurality of force generating components, each force generating component being configured to exert a compression force on a respective compression element of the plurality of compression elements to cause the compression element to compress the fluid tubing against the rigid tubing support surface when the fluid tubing is received by the infusion pump; and a plurality of cams operably connected to the plurality of compression elements, each cam configured to, when rotated, periodically apply a counterforce to a respective compression element to counteract the compression force being applied to the respective compression element and to lift the respective compression element away from compressing the fluid tubing according to a cam profile of the cam, wherein, while the plurality of cams are rotated, the compression forces exerted on the plurality of compression elements, counteracted by each of the compression elements being periodically lifted according to the respective cam profiles of the plurality of cams, create a sinusoidal compression force against the fluid tubing to displace the fluid within the fluid tubing in a downstream direction of flow. Other aspects include corresponding systems and methods for implementation of the corresponding apparatus and its features.

According to various implementations, a machine-implemented method comprises: providing a pumping mechanism for an infusion pump, the pumping mechanism comprising a plurality of compression elements configured to, in a default state, each compress a fluid tubing loaded into the pumping mechanism against a rigid tubing support surface; operating a plurality of cams in an infusion pump to consecutively lift each respective compression element of the plurality of compression elements away from compressing the fluid tubing, against a force being applied by the respective compression element, wherein each of the plurality of compression elements are caused to compress the fluid tubing until lifted by a respective cam of the plurality of cams; and continuously rotating the plurality of cams to create a sinusoidal compression force against the fluid tubing to displace a fluid within the fluid tubing in a downstream direction of flow. Other aspects include corresponding apparatuses and systems for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts a perspective view of an example infusion pump showing an intravenous (IV) infusion set loaded in place within the infusion pump, according to various aspects of the subject technology.

FIG. 2 depicts an example patient care unit shown, according to various aspects of the subject technology.

FIGS. 3A-3C depict example pumping mechanisms of a large volume infusion pump including a pumping mechanism, according to various aspects of the subject technology.

FIG. 4 depicts an example pumping mechanism that utilizes force-driven infusion pumping elements that are operated by an inverted cam profile, according to various aspects of the subject technology.

FIG. 5 depicts an example process for operating an infusion pump that includes force-driven infusion pumping elements, according to aspects of the subject technology.

FIG. 6 is a conceptual diagram illustrating an example electronic system for operating an infusion pump that includes force-driven infusion pumping elements, according to aspects of the subject technology.

### DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology includes a pumping mechanism for an infusion pump that utilizes force-driven infusion pumping elements operated by an inverted cam profile. Each pumping element, or pumping finger, is preloaded by a spring force that pushes the finger to a closed position. The spring force may be implemented as a series of individual springs (e.g., compression, torsion, extension, or leaf springs), a single leaf spring or, in some implementations, a series of opposing magnets. In this manner, each pumping element/finger is biased to apply a default compression force against the pump's platen, and against an IV infusion set when loaded into the pump. The pump's cam is configured with an inverted cam profile that, instead of pushing the fingers into the set, lifts the fingers from the set according to a predetermined pumping cycle. In this regard, the linear finger travel allowed by the cam may be more than that allowed by the set and platen. The cam profile may be adjusted so that there is always an overlap of two or more fingers at the bottom, in an occluding position.

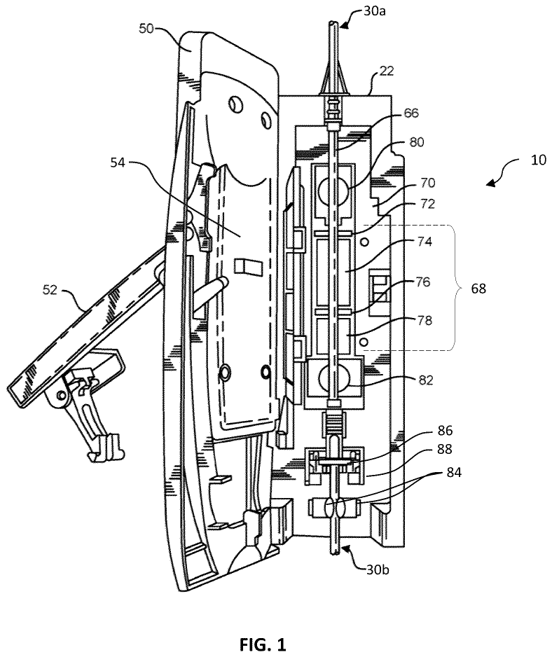

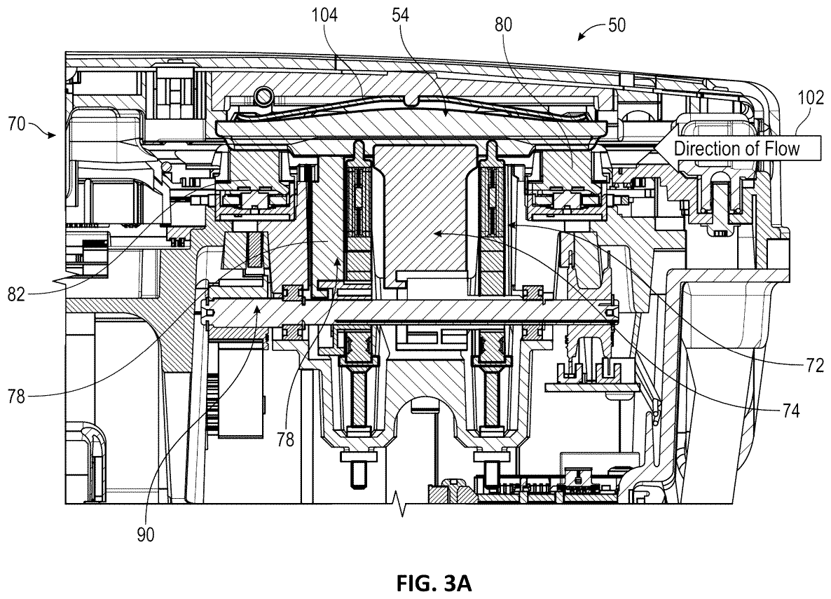

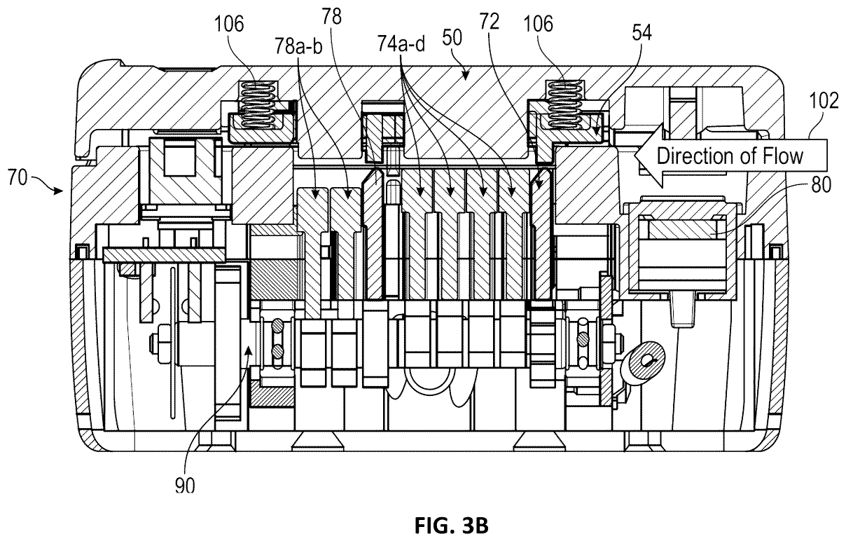

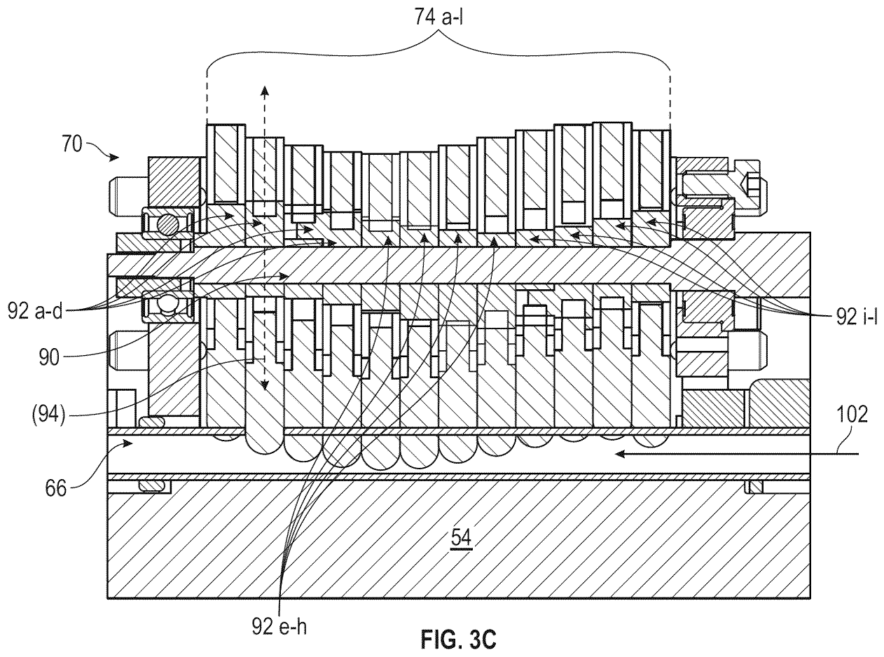

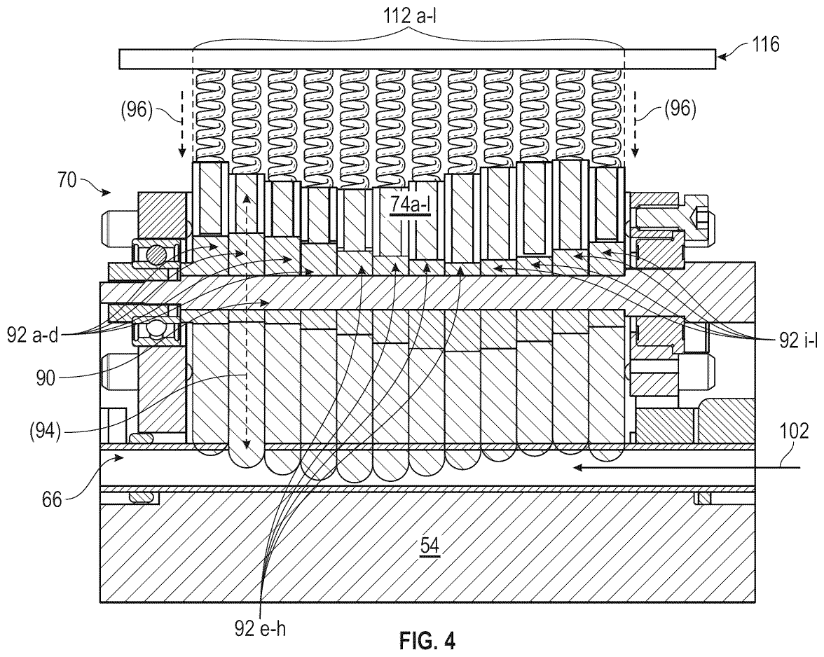

FIG. 1 depicts a perspective view of an example infusion pump showing an intravenous (IV) infusion set 30 loaded in place within the infusion pump, according to various aspects of the subject technology. An infusion system for parenteral infusion of a medical fluid to a patient comprises a pump unit 10, a major part of which comprises a housing or casing 22 which accommodates, in manner known per se, a cam (see FIGS. 3A-3C, 4) controlling a pumping mechanism including a plurality of pumping elements, an electric motor and associated gearing, driving said cam mechanism, and further accommodates electronic control and processing circuitry for controlling such motor and processing signals from pressure sensors etc. provided on the unit. The pump unit, as shown, may also comprise an electronically operated display, an alarm light, an input keyboard or other manually operated controls.

As shown in FIG. 1, the infusion pump 10 (aka an “infusion device”) is shown in perspective view with the front door 50 open, showing the upstream portion 30a of the IV infusion set (e.g., portion between fluid container and the infusion pump 30) and downstream portion 30b (e.g., portion between the infusion pump 10 and a patient) in operative engagement with a pumping mechanism 70 of the infusion pump 10. The infusion set tubing may include a continuous fluid conduit formed from the upstream fluid line 30a to the downstream fluid line 30b, extending from a respective fluid supply to a patient, through which fluid is acted upon by the pump to move fluid downstream to the patient. The infusion pump 10 directly acts on a pumping segment 66 of the infusion set that passes through the pumping mechanism 70 of the pump. Specifically, the pumping mechanism 70 includes a plurality of pumping elements 68 and acts as a flow control device of the pump to move fluid though the conduit. The depicted references 30a-b, 66 may be used to describe herein portions of one continuous fluid line or, in some implementations, may individually describe portions that are fluidly connected together to form a continuous fluid line.

As shown in FIG. 1, the door or face plate 50 which may be opened to reveal the internal loading mechanism for an infusion set. Within the housing of the infusion pump (e.g., behind the door or face place), the infusion pump includes a pumping mechanism configured to operate on and compress an elongated compressible channel of the pumping segment 66, when loaded within the pumping mechanism of the pump. The pumping mechanism 70 includes a group of serially-aligned pumping elements 68 (e.g., occluders 72, 76 and/or pumping fingers 74, 78) configured to compress the elongated compressible channel (e.g., an IV tubing segment) against a rigid tubing support surface 54 when the pumping segment 66 is loaded within the pumping mechanism.

The infusion set includes an intermediate section of resiliently compressible tubing 66, for example of silicone rubber and, in some implementations, upper and/or lower fittings which each tubing section may be connected respectively with a respective upper line 30a and with the lower line 30b. In use, each upper line 30a extends upwardly to a source of the medical fluid to be administered whilst the lower line 30b extends from the infusion pump to an infusion needle or the like inserted into the patient. In use, the compressible tubing 66 (or “pumping segment”) is extended across the face or deck of the pump unit so that its fittings (not labeled) are received in respective brackets respectively and so that the tubing segment extends over a pumping mechanism. In the depicted example, the pumping mechanism 70 includes a four element pump assembly 72, 74, 76, 78. (See also FIG. 3A.) In the depicted example, the infusion set is fitted in place in this fashion whilst the door 50 is in the open position. After the infusion line has been so fitted, the door 50 may be moved to the closed position and is secured by a catch 52 which may include a lever mounted on the outer edge of the door. When the door is closed, the rigid support surface 54 is locked below the pump assembly so that the pumping segment 66 may be compressed against the surface 54 by elements 72-78.

The depicted pumping mechanism 70 includes respective elements 72, 74, 76, 78 (e.g., plungers, fingers, and/or occluders) that are moveable by a cam system inwards and outwards from the face or deck of the pump to compress a respective tubing segment against a platen 54 (e.g., a counter surface or anvil) to propel fluid within the infusion line. In order to make it easier to maintain sterile conditions, these pumping elements may be covered by a thin flexible membrane, (not shown), sealed at its edges with respect to the deck. The pumping elements of the pump assembly periodically press the flexible resilient tubing against the platen 54 which, as described previously, may be configured on an opposite side, for example, on an inner portion of the door 50.

The type of pumping mechanism may vary, including in the number of pumping elements (e.g., pumping fingers. In the depicted example, the pumping mechanism 70 includes an upstream occluding element or occluder 72, a primary pumping element or pump finger 74, a downstream occluding element occluder 76, and a secondary pumping element or flow finger 78. The pumping mechanism (and mechanisms used in other linear peristaltic pumps) operate by sequentially pressing on a segment of the fluid conduit by means of the cam-following pumping elements (e.g., occluders, pumping fingers and flow fingers) 72, 74, 76, and 78, which in the depicted example make a four finger pump assembly. Each element may be sequentially activated by a respective cam lobe on a camshaft to apply a downward compression against the compressible tubing 66 (or pumping segment), to move the fluid in the tubing 66 downstream. Pump finger 74 may include multiple intermediate elements or fingers (see FIG. 3B) that sequentially activate according to positioning of the cam lobes. In some implementations, the pressure is applied in sequential locations of the conduit, beginning at the upstream end of the pumping mechanism, and working toward the downstream end. At least one finger is always pressing hard enough to occlude the conduit. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

An upstream pressure sensor 80 may also be included in the pump 10. The upstream pressure sensor may be mounted to pumping mechanism 70 or located adjacent and upstream in relation to the pumping mechanism 70 between a fluid supply and the pumping mechanism 70, so that the connection of the correct fluid supply with the correct pump may be verified before any fluid is pumped to the patient. A downstream pressure sensor 82 is also included in the example infusion pump 10 at a downstream location with respect to the pumping mechanism, that is, at a location between the patient and the flow control device, so that the connection of the correct fluid supply with the correct pump may be verified before any fluid is pumped to the patient. Downstream pressure sensor 82 may be used to detect a pressure change adjacent to occluder 76 and/or downstream finger 78, to determine whether these elements are functioning properly.

The infusion pump 10 may also include an air-in-line (AIL) sensor 84. In some implementations, air-in-line sensor 84 may be mounted at a bottom end of chassis 22. air-in-line sensor 84 may include two sensing elements disposed on opposite sides of a slot through which the infusion tubing 66 passes. The air-in-line sensor 84 includes a signal (e.g., an ultrasound signal) traveling across the tubing that includes the infusion fluid). Accordingly, infusion tubing 66 should be seated appropriately so that the ultrasound signal in air-in-line sensor 84 traverses the fluid.

In some implementations, the pumping segment 66 may include a cartridge 86 that clips into a compartment 88 of the pump for proper positioning. In the depicted example, the cartridge 86 includes a flow stop that is received into a corresponding compartment 88 in the casing 22 above the air-in-line sensor 84. In this regard, the flow stop cartridge plugs into the compartment and facilitates alignment of the pumping segment 66 with the pumping components, and further facilitates alignment of the downstream line 31 with the air-in-line sensor 84.

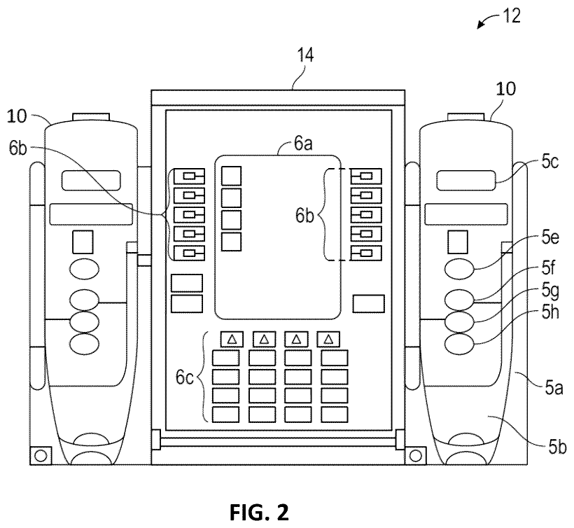

FIG. 2 depicts an example patient care unit 12 shown, according to various aspects of the subject technology. FIG. 2 shows two functional infusion pumps 10 (e.g., “infusion pump modules”) mounted at either side of a main frame infusion controller 14, and the displays and control keys of each, with the main frame infusion controller 14 being capable of programming both infusion pumps. The infusion pump includes a door 5a and a handle 5b that operates to lock the door in a closed position for operation and to unlock and open the door for access to the internal pumping and sensing mechanisms and to load administration sets for the pump. When the door 5a is open, the tube can be connected with the pump 10. When the door 5a is closed, the tube is brought into operating engagement with the pumping mechanism, the upstream and downstream pressure sensors, and the other equipment of the pump. A display 5c, such as an LED display, is located in plain view on the door in this embodiment and may be used to visually communicate various information relevant to the pump 10, such as alert indications (e.g., alarm messages). Control keys 5e-h may exist for programming and controlling operations of the infusion pump as desired. In some implementations, the control keys may be presented as interactive elements on the display 5c (e.g., touchscreen display). The main frame and/or functional module may also include audio alert equipment in the form of a speaker (not shown).

The main frame infusion controller 14 of the patient care unit 12 includes a display 6a for visually communicating various information, such as the operating parameters of a connected pump and alert indications and alert messages, and control keys 6b and 6c for selecting and/or setting control parameters and/or options for controlling the patient care unit 12 and connected modules. The main frame infusion controller 14 may also include a speaker to provide audible alerts. In some implementations, the display 6a may be implemented as a touchscreen display. In such implementations, the control keys 6b may be omitted or reduced in number by providing corresponding interactive elements via a graphical user interface presented via the display 6a. In some implementations, each control key 6b (or 6c) may select a corresponding option displayed in display 6b.

The main frame infusion controller 14 may include a communications system (not shown) with which the main frame infusion controller 14 may communicate with external equipment such as a medical facility server or other computer and with a portable processor, such as a handheld communication device or a laptop-type of computer, or other information device that a clinician may have to transfer information as well as to download drug libraries to a functional module 10. The communication module may be used to transfer access and interaction information for clinicians encountering the main frame infusion controller or device coupled therewith (e.g., pump 10 or bar code scanner). The communications system may include one or more of a radio frequency (RF) system, an optical system such as infrared, a BLUETOOTH™ system, or other wired or wireless system. The bar code scanner and communications system may alternatively be included integrally with the infusion pump 10, such as in cases where a main frame infusion controller is not used, or in addition to one with the main frame infusion controller 14. Further, information input devices need not be hard-wired to medical instruments, information may be transferred through a wireless connection as well. Additionally, other types of modules may be connected to the pump modules or to the main frame infusion controller such as a syringe pump module, patient controlled analgesic module, end tidal CO₂ monitoring module, oximeter monitoring module, or the like.

FIGS. 3A-3C depict example pumping mechanisms 70 of a large volume infusion pump, according to various aspects of the subject technology. While a large volume infusion pump is depicted and used to describe the subject technology, the subject technology may be applied to numerous other pumps that utilize compression of tubing to effect the pumping action.

Infusion devices, such as those that include large volume infusion pumps (LVPs), may use a linear peristaltic or similar type of mechanism that creates the pumping action by way of squeezing the IV tubing to generate the pressure that causes the IV medication to flow through the IV tubing and ultimately into the patient. The squeezing action can also be used as a mechanism to close off the flow. This is used in pumping mechanisms that operate in a cyclical fashion by operating in a cycle that includes a filling portion and a delivery portion.

As depicted in FIGS. 3A and 3B, a typical medical pump for IV infusion delivery may implement valving that includes an intake (or fill) occluder valve 72, and an outflow (or delivery) valve 76. The pumping mechanism 70 may include a single pump finger 74 in between occluders 72, 76, as depicted in FIGS. 1 and 3A, or may include multiple pump fingers, as depicted in FIGS. 3B and 3C. The occluders and plunger/fingers coordinate with each other in programmable, sequential steps, controlled by a cam system 90 to have two phases: 1) a filling phase, and 2) a delivery phase. The occluders and fingers cooperate to compress the pumping segment 66 of the IV infusion set against a rigid platen or support surface 54 to move fluid within the pumping segment 66 (and tubing 30) by sequentially compressing the tubing, thereby causing a flow in a direction 102 according to the particular compression sequence of the occluders.

In FIGS. 3A and 3B, proper operation of the pumping mechanism requires complete closure of the occluder valves 72, 76 when they are not expected to be open. Incomplete closure will produce leakage, which can produce uncontrolled flow resulting in either, not enough medication, or too much medication being delivered to the patient.

Fluid is moved through the IV line 30, 66 using, in implementations according to FIGS. 3A and 3B, the coordinated sequence of actions involving an upstream occluder 72, a pump finger 74, a downstream occluder 76, and a flow finger 78. During the medication infusion process, in the filling phase, the upstream occluder 72 lifts to suck the medication into the tubing segment, which creates a pause, followed by the delivery phase to push the fluid out. In this regard, the upstream occluder 72 compresses the IV line to prevent backflow toward the pumping segment 66, while the downstream occluder 76 remains open, allowing forward flow. The pump finger 74 then compresses the tubing at a specific point, pushing a precise amount of fluid downstream. As the pump finger 74 completes its stroke, the downstream occluder 76 closes to prevent fluid from flowing backward, and the upstream occluder 72 may release to allow fluid to refill the portion of the pumping segment 66 behind pump finger 74.

The flow finger 78 may fine tune the rate of fluid delivery by partially compressing the pumping segment 66 to control the flow as needed. This process operates in a repetitive, tightly synchronized cycle to ensure accurate and consistent fluid delivery. In coordinating the occluders and pump fingers, the pumping mechanism 70 ensures unidirectional flow 102.

In the depicted examples, the platen 54 is integrated into the door 50 of the infusion pump 10. However, the platen 54 may integrated into the pumping mechanism 70 in other implementations (e.g., when the pumping segment 66 loaded from a side of the mechanism). The platen 54 provides a rigid, flat surface that provides support for the tubing, so that a consistent pressure can be applied to the tubing by the pumping occluders and fingers to ensure precise fluid delivery.

According to various implementations, the platen 54 may be spring loaded and/or supported by one or more spring mechanisms. In this manner, the force provided by the spring(s) may counter the occluding force and facilitate an even distribution of pressure along the tubing. In some implementations, as depicted in FIG. 3A, the platen 54 may be supported by one or more leaf springs 104 (e.g., flexible steel or metal alloy). In some implementations, as depicted by FIG. 3B, the platen may be supported by one or more coiled compression springs 106. In this regard, the compression force applied by the occluders and/or fingers may, at least in part, be a function of the force or resistance provided by the spring loaded platen. In some implementations, the amount of preload on the spring elements supporting the platen may be precalibrated, for example, based on the type of administration set materials expected to be used by the pump 10, and the amount of pressure in the set at any given point in time.

Additionally or in the alternative, as depicted in FIG. 3A, each of the depicted occluders 72, 76 may be biased with a respective compression spring 112 which causes the occluder to exert a default compression force on the compressible tubing 66 loaded within the pump 10 and positioned between the platen 54 and the occluders 72, 76. In such implementations, each compression spring 112 places the occluder and/or finger in a compression state by default, while the cam 90 rotates to lift each occluder and/or finger from its default compression state according to a predetermined pumping cycle. In FIG. 3B, the cam motion directly causes the compression state, while the spring-loaded platen 54 provides calibration of the force applied to the tubing therebetween.

In some implementations, a compression force or resistance component of the spring(s) supporting the platen may be adjusted by a mechanical actuator to adjust the occluding force applied to the pumping segment 66. In this regard, the platen 54 may act as a reference surface for the occluders and/or fingers of the pumping mechanism 70, by which a force against the occluders/fingers can be increased to increase the overall compression of the tubing, or reduced to reduce the overall compression of the tubing. In some implementations, a mechanical actuator may adjust the position of the door latch or the hinge (or both) to adjust the overall compression force.

FIG. 3C depicts an example pumping mechanism 70 that includes multiple pumping fingers 74a-l. In the depicted example, occluders 72, 78 are omitted. However, the pumping mechanism of FIG. 3C may include occluders. For example, the example of FIG. 3B may be modified to include the pumping finger configuration of FIG. 3C. The example of FIG. 3C includes twelve pumping fingers 74a-l; however, it is understood that greater than twelve or less than twelve (e.g., four as in FIG. 3B) pumping fingers may be included.

The depicted pumping mechanism operates as a peristaltic pump. A plurality of compression elements (fingers) 74 are configured to compress a flexible fluid tubing 66 filled with a fluid against a platen 54 (a rigid tubing support surface) when the fluid tubing 66 is received by the infusion pump 10 and the pump is activated. In this regard, the depicted fingers 17a-l collectively operate according to a coordinated sequence of compression and release actions to move a fluid through the fluid tubing 66. Each of the fingers 74 a-l may be arranged at equal intervals around a cam assembly 90 (e.g., 30-degrees intervals) that includes corresponding cam lobes 92 a-l (also referred to as cams, herein). Each finger may be mounted on a pivot mechanism and/or be driven by a respective cam 92. As the cam assembly 90 turns, the fingers 74 are caused to cyclically move toward and away (94) from the flexible tubing 66.

The sequential engagement of the fingers 74 (controlled by the cam(s)) creates a traveling wave of compression along the tube's 66 length, moving the fluid through the tube in a predetermined direction of flow (102). When each finger 74 retracts the flexible tube's elastomeric material is allowed to restore to its original shape, generating a vacuum effect that draws additional fluid into the tube. According to various implementations, multiple fingers are in contact with the tube at all times, eliminating gaps in compression that could cause pulsation or flow interruptions. A consistent seal is also maintained, preventing backflow and ensuring precise fluid displacement.

FIG. 4 depicts an example pumping mechanism 70 that utilizes force-driven infusion pumping elements 74 that are operated by an inverted cam profile, according to various aspects of the subject technology. Similar to FIG. 3C, the depicted pumping mechanism 70 includes a plurality of compression elements/fingers 74a-l configured to compress a fluid tubing 66 filled with a fluid against the rigid tubing support surface 54 when the fluid tubing is received by the infusion pump 10. According to various implementations, the depicted pumping mechanism 70 includes a plurality of force generating components 112 a-l, each being configured to exert a compression force on a respective element 74. Each force generating component 112 is configured to exert a compression force (96) on a respective compression element 74 to cause, collectively, the compression elements 74a-l to compress the fluid tubing 66 against the platen 54 when the fluid tubing is received by the infusion pump.

Each respective force generating component 112 may be implemented as a compression spring, as depicted. It is understood that, however, in some implementations the force generating element 112 may include other for generating implementations. For example, in some implementations, each of the plurality of force generating components 112 a-l includes a pair of magnets, with poles of the pair of magnets positioned to exert a mutually repulsive force upon each other. In some implementations, each of the plurality of force generating components 112 includes a leaf spring. In some implementations, each of the leaf springs is a tooth of an integrated comb-like structure supported by a spine that runs along a side of the plurality of compression elements. In a comb implementation, each leaf spring may exert its compression force on each respective compression element 74 from a side of the respective compression element 74.

A cam assembly 90 includes a plurality of cams 92 a-l connected to the plurality of compression elements 74 a-l. Each cam 92 configured to, when rotated, periodically apply a counterforce to a respective compression element 74 to counteract the compression force applied to the respective compression element 74 and lift the respective compression element 74 away from compressing the fluid tubing 66 according to a cam profile of the cam 92. According to various implementations, the cam profile is inverted with respect to conventional cam profiles such that, instead of pushing the pump's fingers 74 into the infusion tubing 66, the fingers 74 are pulled away from the tubing, against the default compression force (96) being applied on the fingers by the force generating component 112. Accordingly, while the plurality of cams are rotated, the compression forces (96) exerted on the plurality of compression elements 74 a-l, are counteracted by each of the compression elements being lifted according to the cam profiles of the plurality of cams. create a sinusoidal compression force against the fluid tubing to displace the fluid within the fluid tubing in a downstream direction of flow 102.

The cams 92 a-l are arranged to create a continuous sinusoidal compression force against the fluid tubing. Each cam of the cam assembly (e.g., camshaft) may be offset from an adjacent cam by a number of degrees equal to 360 divided by a number of the cams. For example, each cam may be offset by 30-degrees in a cam assembly with 12 cams and 12 fingers. According to various implementations, each cam 92, when rotated, lifts the respective compression element 74 momentarily away from the fluid tubing 66 for a first portion of a rotation cycle (against the force of the force generating component), and then releases the respective compression element 74 for a second portion of the rotation cycle to allow the force generating component 112 associated with the respective compression element 74 to exert the compression force on the respective compression element 74 and cause the respective compression element 74 to compress the fluid tubing 66. A first cam 92a may cause a first compression element 74a to be released while a second cam 92b applies the counterforce to a second compression element 74b to lift the second compression element 74b away from the fluid tubing 66.

In some implementations, the plurality of cams 92 a-l, when rotated when rotated according to the offset pattern, are configured to cause any two adjacent compression elements 74 to be simultaneously released to simultaneously compress and occlude the fluid tubing 66 for a period of the rotation. This may then be followed by a downstream one of the adjacent compression elements 74 being lifted while the other adjacent compression element 74 remains released to compress and occlude the fluid tubing 66. In this manner, there may always be an overlap of two or more fingers 74 at the bottom, momentarily occluding the tube 66 at the same time. Accordingly, a more consistent flow rate is generated and the sensitivity to back pressure and/or back flow is reduced.

According to various implementations, each respective compression spring 112 may be positioned between a support reference 116 and each compression element 74, causing each element 74 to exert a default compression force on the fluid tubing. In such implementations, the support reference 116 may include a fixed part of the pump mechanism's chassis (not shown), or may be movable, for example by an actuator (not shown), such that it's position with regard to the cam assembly 90 and/or tubing 66 may be adjusted. In this manner, the support reference 116 acts as a reference surface for the sprung elements, and an adjustment to its position changes a pre-load compression on the compression springs 112.

In some implementations, the actuator device includes a motor controlled mechanism that directly acts on the support reference 116 to adjust it's location relative to the tubing loaded within the pump (e.g., vertical position relative to the horizontally positioned tubing). During a pumping operation, the actuator may cause the support reference 116 to move away in an opposite direction from the tubing so as to reduce the preload on the compression springs and thus reduce compression force exerted by the pumping elements 68 on the fluid tubing, thereby extending the life of the tubing.

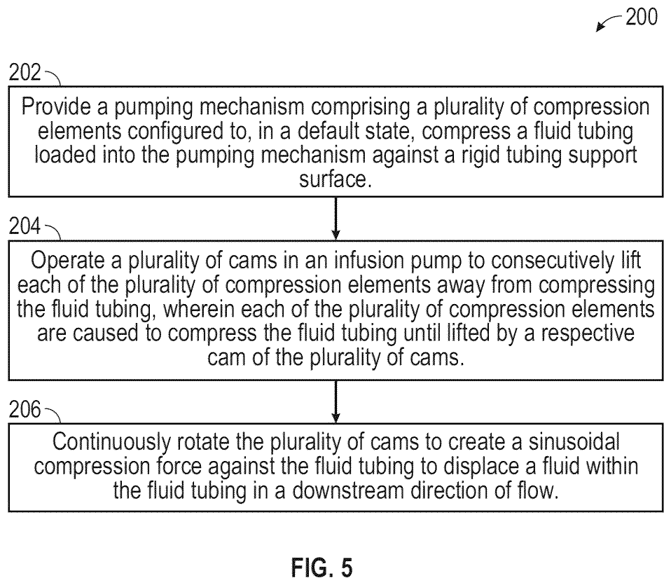

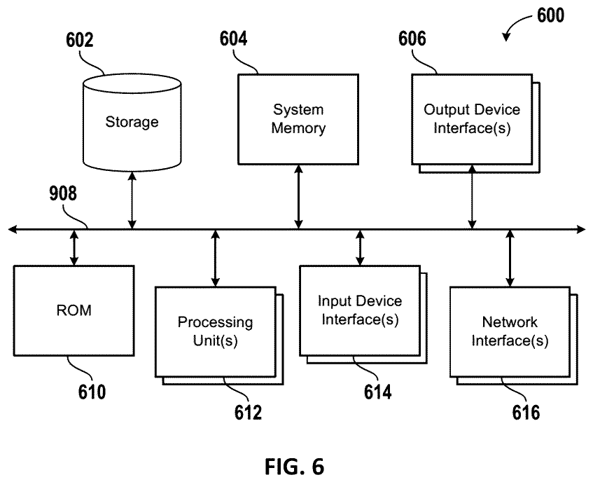

FIG. 5 depicts an example process 200 for operating an infusion pump that includes force-driven infusion pumping elements, according to aspects of the subject technology. For explanatory purposes, the various blocks of example process 200 are described herein with reference to FIGS. 1 through 4, and the components and/or processes described herein. The one or more of the blocks of process 200 may be implemented, for example, by the infusion pump 10 described herein. For example, one or more of the blocks may be implemented based on electromechanical and/or computer control by the pump's electronic subsystems (see, e.g., FIG. 6). For explanatory purposes, the blocks of example process 200 are described as occurring in serial, or linearly. However, the blocks of example process 200 may occur in parallel. In addition, the blocks of example process 200 need not be performed in the order shown and/or one or more of the blocks of example process 200 need not be performed.

As shown in FIGS. 3A-3C and 4, a pumping mechanism is provided (202) that includes a plurality of compression elements 74 a-l (see, e.g., FIG. 4) configured to, in a default state, compress a fluid tubing 66 loaded into the pumping mechanism against a rigid tubing support surface. According to various implementations, the pumping mechanism 70 is provided as part of an infusion pump 10. In some implementations, the pumping mechanism is provided apart from the infusion pump, for example, as a component that may be configured to work in association with the infusion pump. In some implementations, the pumping mechanism is integrated within the infusion pump (e.g., during a manufacturing process).

According to the depicted example, a controller of the infusion pump 10 (e.g., main frame infusion controller 14 or a processor therein or within the pump module), in connection with a pumping mechanism, causes one or more pumping elements to periodically compress a fluid tubing (e.g., pumping segment 66) against a rigid support surface 54 to pump a fluid through the fluid tubing in a predetermined direction of flow 102. In this regard, a plurality of cams are operated to consecutively lift each respective compression element 74 of the plurality of compression elements away from compressing the fluid tubing (202). Each compression element 74 is lifted against a force being applied to the respective compression element 74 by a respective force generating component 112. In this manner, each of the plurality of compression elements 74 is caused to compress the fluid tubing until lifted by a respective cam of the plurality of cams.

As described previously, the plurality of compression elements 74 a-l may be caused to compress the fluid tubing by way of a plurality of force generating components 112 a-l acting upon the compression elements 74. Accordingly, each force generating component 112 may be configured to exert a respective compression force on a respective compression element 74, such that when each cam is rotated, the cam periodically applies a counterforce to a respective compression element to counteract the compression force being applied to the respective compression element and lift the respective compression element away from compressing the fluid tubing 66 according to a cam profile of the cam.

In some implementations, each cam is rotated to lift a respective compression element 74 momentarily away from the fluid tubing 66 for a first portion of a rotation cycle, and then release the respective compression element for a second portion of the rotation cycle to allow the force generating component associated with the respective compression element to exert the compression force on the respective compression element and cause the respective compression element to compress the fluid tubing. Accordingly, a first cam may be caused to release a first compression element while a second cam applies the counterforce to a second compression element to lift the second compression element away from the fluid tubing.

As described previously, the controller may rotate the plurality of cams to cause any two adjacent compression elements to be simultaneously released to simultaneously compress and occlude the fluid tubing for a period of the rotation, followed by a downstream one of the adjacent compression elements being lifted while the other adjacent compression element remains released to compress and occlude the fluid tubing.

As depicted, the plurality of cams are continuously rotated to create a sinusoidal compression force against the fluid tubing to displace a fluid within the fluid tubing in a downstream direction of flow (206).

Many of the above-described devices, systems and methods, may also be controlled by software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 6 is a conceptual diagram illustrating an example electronic system 600 for operating an infusion pump that includes force-driven infusion pumping elements, according to aspects of the subject technology. Electronic system 600 may be representative of a control unit and/or computing device for execution of software associated with one or more components and processes provided by FIGS. 1 through 5, including but not limited to infusion pump 10 (e.g., a processing system of controller 14 or within infusion pump 10). Electronic system 600 may be representative of a device used in connection or combination with the disclosure regarding FIGS. 1 through 5. In this regard, electronic system 600 may be a device connected to the infusion pump 10, for example, to activate the occluders and/or pumping fingers, the cam of the pumping mechanism, or to monitor or control same. For example, system 600 may be representative of a personal computer or a mobile device such as a smartphone, tablet computer, laptop, personal digital assistant (PDA), an augmented reality device, a wearable such as a watch or band or glasses, or combination thereof, or other touch screen or television with one or more processors embedded therein or coupled thereto, or any other sort of computer-related electronic device having network connectivity specifically configured to implement one or more of the features described.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and processes previously described.

Bus 608 collectively represents all system, peripheral, and chipset buses that communicatively connect the numerous internal devices of electronic system 600. For instance, bus 608 communicatively connects processing unit(s) 612 with ROM 610, system memory 604, and permanent storage device 602.

From these various memory units, processing unit(s) 612 retrieves specific instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random-access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process, in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, a personal area network (“PAN”), or an Intranet, or a network of networks, such as the Internet. Any or all components of electronic system 600 can be used in conjunction with the subject disclosure.

The functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of specific instructions for performing various operations described herein. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs) specifically configured with one or more of the features described. In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a specifically configured computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of specifically configured devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a specifically configured computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other (e.g., physically separated) and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An infusion pump, comprising: a plurality of compression elements configured to compress a fluid tubing filled with a fluid against a rigid tubing support surface when the fluid tubing is received by the infusion pump; a plurality of force generating components, each force generating component being configured to exert a compression force on a respective compression element of the plurality of compression elements to cause the compression element to compress the fluid tubing against the rigid tubing support surface when the fluid tubing is received by the infusion pump; and a plurality of cams operably connected to the plurality of compression elements, each cam configured to, when rotated, periodically apply a counterforce to a respective compression element to counteract the compression force being applied to the respective compression element and to lift the respective compression element away from compressing the fluid tubing according to a cam profile of the cam, wherein, while the plurality of cams are rotated, the compression forces exerted on the plurality of compression elements, counteracted by each of the compression elements being periodically lifted according to the respective cam profiles of the plurality of cams, create a sinusoidal compression force against the fluid tubing to displace the fluid within the fluid tubing in a downstream direction of flow.

Clause 2. The infusion pump of Clause 1, further comprising: a processor configured to: activate the plurality of cams to create the sinusoidal compression force against the fluid tubing.

Clause 3. The infusion pump of Clause 1 or Clause 2, wherein an amount by which the fluid tubing is compressed against the rigid tubing support surface by each compression element is based on an amount of the compression force exerted by the force generating component acting on the compression element.

Clause 4. The infusion pump of any one of Clauses 1-3, wherein the plurality of force generating components comprise a plurality of compression springs.

Clause 5. The infusion pump of any one of Clauses 1-3, wherein the plurality of force generating components comprise a plurality of leaf springs.

Clause 6. The infusion pump of Claim 5, wherein each of the plurality of leaf springs are integrated into a comb-like structure as teeth supported by a spine that runs along a side of the plurality of compression elements, with each leaf spring exerting the compression force on each respective compression element from a side of the respective compression element.

Clause 7. The infusion pump of any one of Clauses 1-3, wherein each of the plurality of force generating components comprises a pair of magnets, with poles of the pair of magnets positioned to exert a mutually repulsive force upon each other.

Clause 8. The infusion pump of any one of Clauses 1-7, wherein the plurality of cams comprise a camshaft, the camshaft being rotated to cause the cams to be rotated according to an offset pattern.

Clause 9. The infusion pump of Claim 8, wherein each cam of the camshaft is offset from an adjacent cam by a number of degrees equal to 360 divided by a number of the plurality of cams.

Clause 10. The infusion pump of Claim 9, wherein each cam, when rotated, lifts the respective compression element momentarily away from the fluid tubing for a first portion of a rotation cycle, and then releases the respective compression element for a second portion of the rotation cycle to allow the force generating component associated with the respective compression element to exert the compression force on the respective compression element and cause the respective compression element to compress the fluid tubing.

Clause 11. The infusion pump of Claim 10, wherein a first cam causes a first compression element to be released while a second cam applies the counterforce to a second compression element to lift the second compression element away from the fluid tubing.

Clause 12. The infusion pump of Claim 11, wherein the plurality of cams, when rotated when rotated according to the offset pattern, are configured to cause any two adjacent compression elements to be simultaneously released to simultaneously compress and occlude the fluid tubing for a period of the rotation, followed by a downstream one of the adjacent compression elements being lifted while the other adjacent compression element remains released to compress and occlude the fluid tubing.

Clause 13. A machine-implemented method, comprising: providing a pumping mechanism for an infusion pump, the pumping mechanism comprising a plurality of compression elements configured to, in a default state, each compress a fluid tubing loaded into the pumping mechanism against a rigid tubing support surface; operating a plurality of cams in an infusion pump to consecutively lift each respective compression element of the plurality of compression elements away from compressing the fluid tubing, against a force being applied by the respective compression element, wherein each of the plurality of compression elements are caused to compress the fluid tubing until lifted by a respective cam of the plurality of cams; and continuously rotating the plurality of cams to create a sinusoidal compression force against the fluid tubing to displace a fluid within the fluid tubing in a downstream direction of flow.

Clause 14. The machine-implemented method of Clause 13, further comprising: causing the plurality of compression elements to compress the fluid tubing using a plurality of force generating components, each force generating component being configured to exert a compression force on a respective compression element of the plurality of compression elements, wherein each cam, when rotated, periodically applies a counterforce to a respective compression element to counteract the compression force being applied to the respective compression element and lift the respective compression element away from compressing the fluid tubing according to a cam profile of the cam.

Clause 15. The machine-implemented method of Claim 14, further comprising: rotating each cam to lift a respective compression element momentarily away from the fluid tubing for a first portion of a rotation cycle, and then release the respective compression element for a second portion of the rotation cycle to allow the force generating component associated with the respective compression element to exert the compression force on the respective compression element and cause the respective compression element to compress the fluid tubing.

Clause 16. The machine-implemented method of Claim 15, further comprising: causing a first cam to release a first compression element while a second cam applies the counterforce to a second compression element to lift the second compression element away from the fluid tubing.

Clause 17. The machine-implemented method of Claim 16, further comprising: rotating the plurality of cams to cause any two adjacent compression elements to be simultaneously released to simultaneously compress and occlude the fluid tubing for a period of the rotation, followed by a downstream one of the adjacent compression elements being lifted while the other adjacent compression element remains released to compress and occlude the fluid tubing.

Clause 18. A peristaltic pump system for pumping a fluid through an infusion set, comprising: a plurality of compression elements configured to compress a fluid tubing filled with a fluid against a rigid tubing support surface when the fluid tubing is received by an infusion pump; a plurality of force generating components, each force generating component being configured to exert a compression force on a respective compression element of the plurality of compression elements to cause the plurality of compression elements to compress the fluid tubing against the rigid tubing support surface when the fluid tubing is received by the infusion pump; a plurality of cams operably connected to the plurality of compression elements, each cam configured to, when rotated, periodically apply a counterforce to a respective compression element to counteract the compression force being applied to the respective compression element and lift the respective compression element away from compressing the fluid tubing according to a cam profile of the cam, wherein, while the plurality of cams are rotated, the compression forces exerted on the plurality of compression elements, counteracted by each of the compression elements being lifted according to the cam profiles of the plurality of cams, create a sinusoidal compression force against the fluid tubing to displace the fluid within the fluid tubing in a downstream direction of flow.

Clause 19. The peristaltic pump system of Clause 18, wherein each cam, when rotated, lifts the respective compression element momentarily away from the fluid tubing for a first portion of a rotation cycle, and then releases the respective compression element for a second portion of the rotation cycle to allow the force generating component associated with the respective compression element to exert the compression force on the respective compression element and cause the respective compression element to compress the fluid tubing, wherein a first cam causes a first compression element to be released while a second cam applies the counterforce to a second compression element to lift the second compression element away from the fluid tubing.

20.The peristaltic pump system of Clause 19, wherein the plurality of cams, when rotated when rotated according to an offset pattern, are configured to cause any two adjacent compression elements to be simultaneously released to simultaneously compress and occlude the fluid tubing for a period of the rotation, followed by a downstream one of the adjacent compression elements being lifted while the other adjacent compression element remains released to compress and occlude the fluid tubing.

### Further Consideration

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. An infusion pump, comprising:
a plurality of compression elements configured to compress a fluid tubing filled with a fluid against a rigid tubing support surface when the fluid tubing is received by the infusion pump;
a plurality of force generating components, each force generating component being configured to exert a compression force on a respective compression element of the plurality of compression elements to cause the compression element to compress the fluid tubing against the rigid tubing support surface when the fluid tubing is received by the infusion pump; and
a plurality of cams operably connected to the plurality of compression elements, each cam configured to, when rotated, periodically apply a counterforce to a respective compression element to counteract the compression force being applied to the respective compression element and to lift the respective compression element away from compressing the fluid tubing according to a cam profile of the cam,
wherein, while the plurality of cams are rotated, the compression forces exerted on the plurality of compression elements, counteracted by each of the compression elements being periodically lifted according to the respective cam profiles of the plurality of cams, create a sinusoidal compression force against the fluid tubing to displace the fluid within the fluid tubing in a downstream direction of flow.

2. The infusion pump of claim 1, further comprising:
a processor configured to:
activate the plurality of cams to create the sinusoidal compression force against the fluid tubing.

3. The infusion pump of claim 1, wherein an amount by which the fluid tubing is compressed against the rigid tubing support surface by each compression element is based on an amount of the compression force exerted by the force generating component acting on the compression element.

4. The infusion pump of claim 1, wherein the plurality of force generating components comprise a plurality of compression springs.

5. The infusion pump of claim 1, wherein the plurality of force generating components comprise a plurality of leaf springs.

6. The infusion pump of claim 5, wherein each of the plurality of leaf springs are integrated into a comb-like structure as teeth supported by a spine that runs along a side of the plurality of compression elements, with each leaf spring exerting the compression force on each respective compression element from a side of the respective compression element.

7. The infusion pump of claim 1, wherein each of the plurality of force generating components comprises a pair of magnets, with poles of the pair of magnets positioned to exert a mutually repulsive force upon each other.

8. The infusion pump of claim 1, wherein the plurality of cams comprise a camshaft, the camshaft being rotated to cause the cams to be rotated according to an offset pattern.

9. The infusion pump of claim 8, wherein each cam of the camshaft is offset from an adjacent cam by a number of degrees equal to 360 divided by a number of the plurality of cams.

10. The infusion pump of claim 9, wherein each cam, when rotated, lifts the respective compression element momentarily away from the fluid tubing for a first portion of a rotation cycle, and then releases the respective compression element for a second portion of the rotation cycle to allow the force generating component associated with the respective compression element to exert the compression force on the respective compression element and cause the respective compression element to compress the fluid tubing.

11. The infusion pump of claim 10, wherein a first cam causes a first compression element to be released while a second cam applies the counterforce to a second compression element to lift the second compression element away from the fluid tubing.

12. The infusion pump of claim 11, wherein the plurality of cams, when rotated when rotated according to the offset pattern, are configured to cause any two adjacent compression elements to be simultaneously released to simultaneously compress and occlude the fluid tubing for a period of the rotation, followed by a downstream one of the adjacent compression elements being lifted while the other adjacent compression element remains released to compress and occlude the fluid tubing.

13. A machine-implemented method, comprising:
providing a pumping mechanism for an infusion pump, the pumping mechanism comprising a plurality of compression elements configured to, in a default state, each compress a fluid tubing loaded into the pumping mechanism against a rigid tubing support surface;
operating a plurality of cams in the infusion pump to consecutively lift each respective compression element of the plurality of compression elements away from compressing the fluid tubing, against a force being applied by the respective compression element, wherein each of the plurality of compression elements are caused to compress the fluid tubing until lifted by a respective cam of the plurality of cams; and
continuously rotating the plurality of cams to create a sinusoidal compression force against the fluid tubing to displace a fluid within the fluid tubing in a downstream direction of flow.

14. The machine-implemented method of claim 13, further comprising:
causing the plurality of compression elements to compress the fluid tubing using a plurality of force generating components, each force generating component being configured to exert a compression force on a respective compression element of the plurality of compression elements, wherein each cam, when rotated, periodically applies a counterforce to a respective compression element to counteract the compression force being applied to the respective compression element and lift the respective compression element away from compressing the fluid tubing according to a cam profile of the cam.

15. The machine-implemented method of claim 14, further comprising:
rotating each cam to lift a respective compression element momentarily away from the fluid tubing for a first portion of a rotation cycle, and then release the respective compression element for a second portion of the rotation cycle to allow the force generating component associated with the respective compression element to exert the compression force on the respective compression element and cause the respective compression element to compress the fluid tubing.

16. The machine-implemented method of claim 15, further comprising:
causing a first cam to release a first compression element while a second cam applies the counterforce to a second compression element to lift the second compression element away from the fluid tubing.

17. The machine-implemented method of claim 16, further comprising:
rotating the plurality of cams to cause any two adjacent compression elements to be simultaneously released to simultaneously compress and occlude the fluid tubing for a period of the rotation, followed by a downstream one of the adjacent compression elements being lifted while the other adjacent compression element remains released to compress and occlude the fluid tubing.

18. A peristaltic pump system for pumping a fluid through an infusion set, comprising:
a plurality of compression elements configured to compress a fluid tubing filled with a fluid against a rigid tubing support surface when the fluid tubing is received by an infusion pump;
a plurality of force generating components, each force generating component being configured to exert a compression force on a respective compression element of the plurality of compression elements to cause the plurality of compression elements to compress the fluid tubing against the rigid tubing support surface when the fluid tubing is received by the infusion pump;
a plurality of cams operably connected to the plurality of compression elements, each cam configured to, when rotated, periodically apply a counterforce to a respective compression element to counteract the compression force being applied to the respective compression element and lift the respective compression element away from compressing the fluid tubing according to a cam profile of the cam,
wherein, while the plurality of cams are rotated, the compression forces exerted on the plurality of compression elements, counteracted by each of the compression elements being lifted according to the cam profiles of the plurality of cams, create a sinusoidal compression force against the fluid tubing to displace the fluid within the fluid tubing in a downstream direction of flow.

19. The peristaltic pump system of claim 18, wherein each cam, when rotated, lifts the respective compression element momentarily away from the fluid tubing for a first portion of a rotation cycle, and then releases the respective compression element for a second portion of the rotation cycle to allow the force generating component associated with the respective compression element to exert the compression force on the respective compression element and cause the respective compression element to compress the fluid tubing, wherein a first cam causes a first compression element to be released while a second cam applies the counterforce to a second compression element to lift the second compression element away from the fluid tubing.

20. The peristaltic pump system of claim 19, wherein the plurality of cams, when rotated when rotated according to an offset pattern, are configured to cause any two adjacent compression elements to be simultaneously released to simultaneously compress and occlude the fluid tubing for a period of the rotation, followed by a downstream one of the adjacent compression elements being lifted while the other adjacent compression element remains released to compress and occlude the fluid tubing.
